(19) United States
(12) Patent Application Publication
(10) Pub. No.: US 2018/0344380 A1
(43) Pub. Date: Dec. 6, 2018

(54) ENERGY TREATMENT TOOL

(71) Applicant: OLYMPUS CORPORATION, Tokyo (JP)

(72) Inventors: Akira MATSUI, Tokyo (JP); Shoei TSURUTA, Tokyo (JP)

(73) Assignee: OLYMPUS CORPORATION, Tokyo (JP)

(21) Appl. No.: 16/047,501

(22) Filed: Jul. 27, 2018

Related U.S. Application Data
(63) Continuation of application No. PCT/JP2016/059678, filed on Mar. 25, 2016.

(51) Int. Cl. A61B 18/10; A61B 18/08

(52) U.S. Cl. CPC A61B 18/10; A61B 2018/00095; A61B 18/085

(57) ABSTRACT

An energy treatment tool includes: a heat transfer plate including a treatment surface for treating body tissue; an energy generating unit configured to generate thermal energy and transmit the thermal energy to the heat transfer plate; and a wiring unit connected to the energy generating unit and serving as an electrical conduction path to the energy generating unit. The wiring unit includes: a low thermal resistance unit; and a high thermal resistance unit that has higher thermal resistance than the low thermal resistance unit and connects the low thermal resistance unit and the energy generating unit.

### CROSS-REFERENCE TO RELATED APPLICATION

This application is a continuation of International Application No. PCT/JP2016/059678, filed on Mar. 25, 2016, the entire contents of which are incorporated herein by reference.

### BACKGROUND

The present disclosure relates to an energy treatment tool.

In the related art, an energy treatment tool (thermal tissue manipulation system) for treatment (connection (or anastomosis), dissection, or the like) of body tissue by applying thermal energy to the body tissue has been known (for example, see JP 2012-24583 A).

The energy treatment tool described in JP 2012-24583 A includes a pair of jaws for grasping body tissue. A resistance heating element is embedded in each of the jaws as a pair. When electricity is supplied to the resistance heating elements via a cable, the jaws are heated and thermal energy is applied to body tissue that is in contact with the jaws.

### SUMMARY

An energy treatment tool according to one aspect of the present disclosure includes: a heat transfer plate including a treatment surface for treating body tissue; an energy generating unit configured to generate thermal energy and transmit the thermal energy to the heat transfer plate; and a wiring unit connected to the energy generating unit and serving as an electrical conduction path to the energy generating unit, the wiring unit including: a low thermal resistance unit; and a high thermal resistance unit that has higher thermal resistance than the low thermal resistance unit and connects the low thermal resistance unit and the energy generating unit.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a treatment system according to a first embodiment;

FIG. 2 is an enlarged view of a tip portion of an energy treatment tool illustrated in FIG. 1;

FIG. 3 is a perspective view illustrating a configuration of an energy applying structure illustrated in FIG. 2;

FIG. 4 is an exploded perspective view of the energy applying structure illustrated in FIG. 3;

FIG. 5 is a diagram of the energy applying structure illustrated in FIG. 3 when viewed from a back side of a treatment surface;

FIG. 6 is a side view of the energy applying structure illustrated in FIG. 3;

FIG. 7 is a diagram illustrating a configuration of an energy applying structure according to a second embodiment;

FIG. 8 is a diagram illustrating a configuration of an energy applying structure according to a third embodiment;

FIG. 9 is a diagram illustrating a configuration of an energy applying structure according to a fourth embodiment; and

FIG. 10 is a diagram illustrating a circuit model of a wire relay unit illustrated in FIG. 9.

### DETAILED DESCRIPTION

Hereinafter, modes (hereinafter, referred to as “embodiments”) for carrying out the present disclosure will be described with reference to the drawings. The present disclosure is not limited by the embodiments below. The same components will be denoted by the same reference signs throughout the drawings.

### First Embodiment

Schematic Configuration of Energy Treatment System

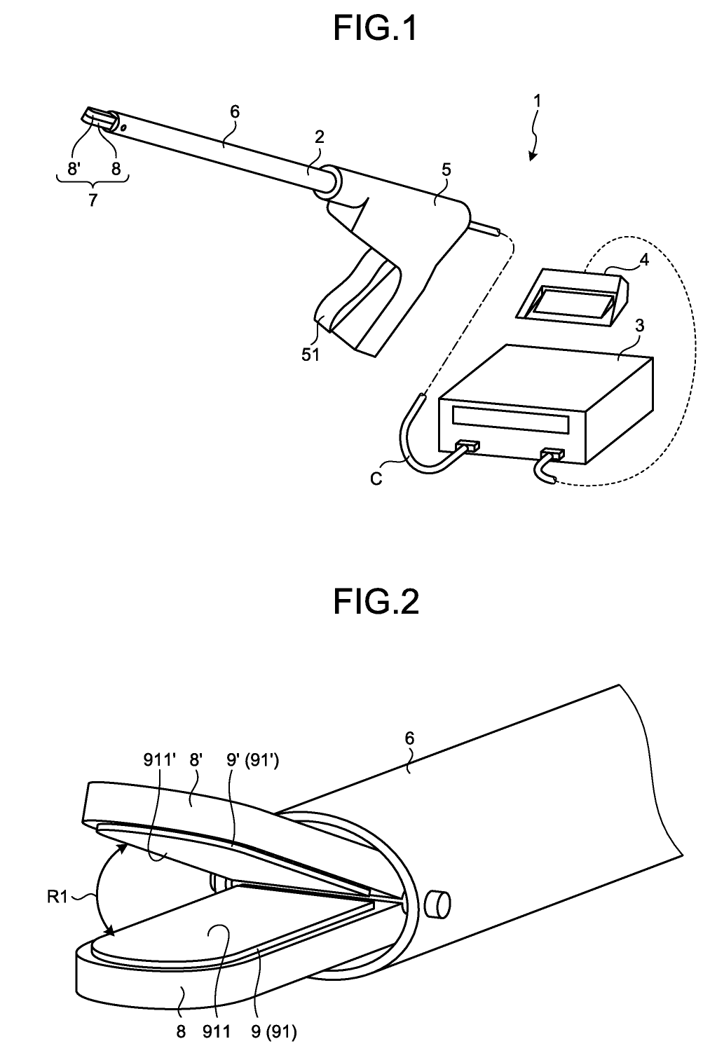

FIG. 1 is a diagram schematically illustrating a treatment system 1 according to a first embodiment.

The treatment system 1 applies energy to body tissue to be treated, and performs treatment (connection (or anastomosis), dissection, or the like) on the body tissue. As illustrated in FIG. 1, the treatment system 1 includes an energy treatment tool 2, a control device 3, and a foot switch 4.

Configuration of Energy Treatment Tool

The energy treatment tool 2 is, for example, a linear-type surgical medical treatment tool for performing treatment on body tissue through an abdominal wall. As illustrated in FIG. 1, the energy treatment tool 2 includes a handle 5, a shaft 6, and a grasping unit 7.

The handle 5 is a part to be held by an operator. As illustrated in FIG. 1, an operation knob 51 is provided on the handle 5.

As illustrated in FIG. 1, the shaft 6 has a substantially cylindrical shape, and one end thereof (a right end portion in FIG. 1) is connected to the handle 5. The grasping unit 7 is mounted on the other end (a left end portion in FIG. 1) of the shaft 6. An opening/closing mechanism (not illustrated) that opens and closes holding members 8 and 8′ (FIG. 1), which constitute the grasping unit 7, in accordance with operation on the operation knob 51 performed by the operator is provided inside the shaft 6. An electrical cable C (FIG. 1) connected to the control device 3 is provided inside the shaft 6 from the one end side to the other end side via the handle 5 connected to the control device 3.

Configuration of Grasping Unit

FIG. 2 is an enlarged view of a tip portion of the energy treatment tool 2.

In FIG. 1 and FIG. 2, a component denoted by a reference sign without “′” and a component denoted by a reference sign with “′” have the same configuration.

The grasping unit 7 is a part for grasping body tissue and performing treatment on the body tissue. As illustrated in FIG. 1 or FIG. 2, the grasping unit 7 includes the pair of holding members 8 and 8′ and a pair of energy applying structures 9 and 9′.

The holding members 8 and 8′ as a pair are pivotally supported on the other end of the shaft 6 (a left end portion in FIG. 2) such that the holding members 8 and 8′ can be opened and closed in directions of arrows R1 (FIG. 2), and are opened and closed in the directions of the arrows R1 in accordance with operation on the operation knob 51 performed by the operator. The holding members 8 and 8′ are made by molding a resin material (fluororesin or the like), for example.

The energy applying structures 9 and 9′ have the same configuration, and are respectively supported, in vertically opposite postures, on an upper surface of the holding member 8 provided in the lower side and on a lower surface of the holding member 8′ provided in the upper side in FIG. 1 and FIG. 2. Therefore, in the following, only a configuration of the energy applying structure 9 supported by the holding member 8 will be described.

Configuration of Energy Applying Mechanism

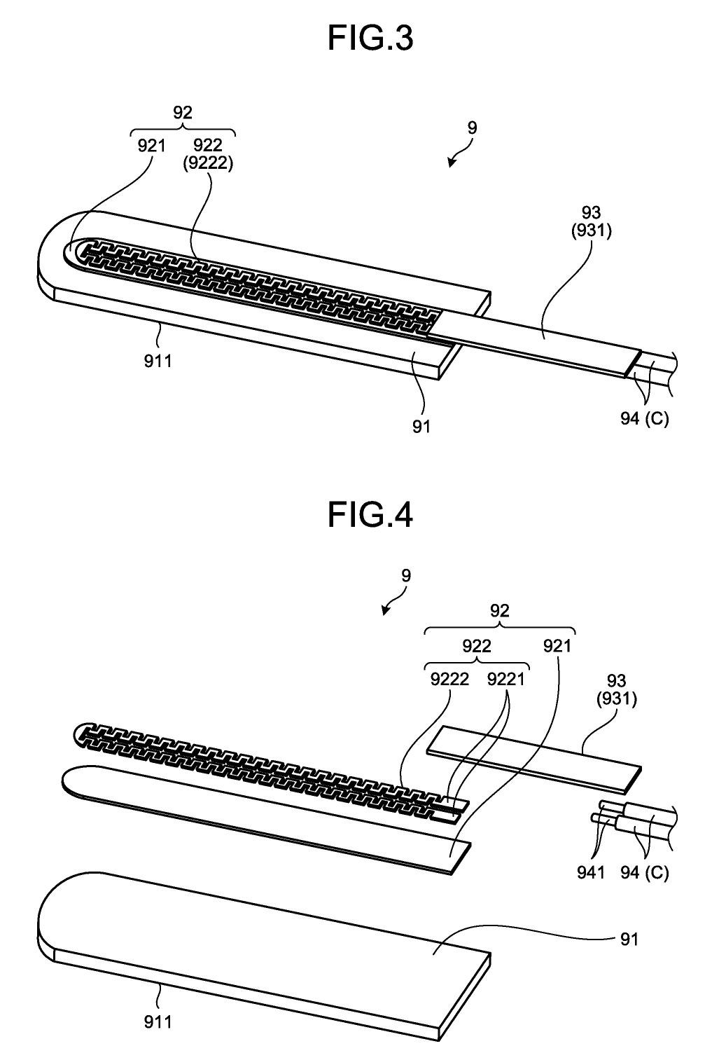

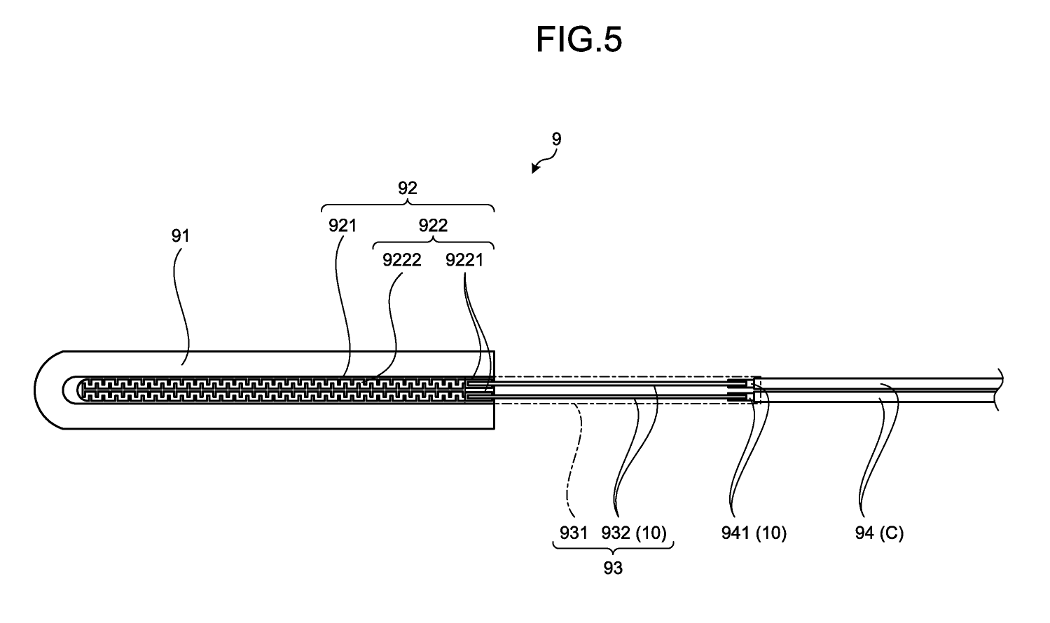

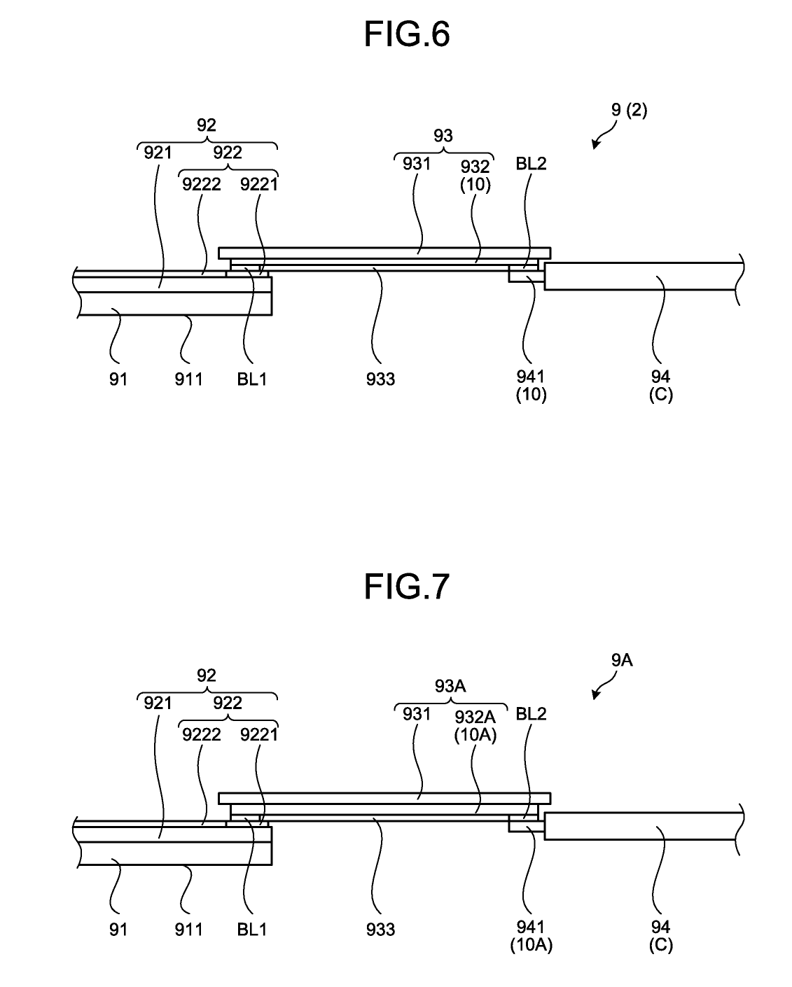

FIG. 3 is a perspective view illustrating the configuration of the energy applying structure 9. FIG. 4 is an exploded perspective view of the energy applying structure 9 illustrated in FIG. 3. FIG. 5 is a diagram of the energy applying structure 9 illustrated in FIG. 3 when viewed from a back side of a treatment surface 911. FIG. 6 is a side view of the energy applying structure 9 illustrated in FIG. 3.

The energy applying structure 9 applies thermal energy to body tissue under the control of the control device 3. As illustrated in FIG. 3 to FIG. 6, the energy applying structure 9 includes a heat transfer plate 91, a ceramic heater 92, a wire relay unit 93, and a pair of lead wires 94 included in the electrical cable C.

The heat transfer plate 91 is an elongated thin plate (an elongated shape extending in a horizontal direction in FIG. 3 to FIG. 6), and, in a state where the energy applying structure 9 is mounted on the holding member 8, the treatment surface 911 that is one plate surface faces the holding member 8′ side (the upper side in FIG. 1 and FIG. 2). Examples of a material of the heat transfer plate 91 include a ceramic material with high thermal conductivity, such as aluminum nitride, and a metallic material with high thermal conductivity, such as copper or aluminum. The treatment surface 911 comes in contact with body tissue while the grasping unit 7 is grasping the body tissue, and the heat transfer plate 91 transfers heat from the ceramic heater 92 to the body tissue (applies thermal energy to the body tissue).

The ceramic heater 92 generates thermal energy, and transfers the thermal energy to the heat transfer plate 91 (heats the heat transfer plate 91). That is, the ceramic heater 92 has a function as an energy generating unit according to the present disclosure. The ceramic heater 92 includes a ceramic substrate 921 and a heating element 922 as illustrated in FIG. 3 to FIG. 6.

The ceramic substrate 921 is an elongated plate made of a ceramic material with high thermal conductivity, such as aluminum nitride or alumina.

Here, a width size and a length size (a length size in the horizontal direction in FIG. 3 to FIG. 6) of the ceramic substrate 921 are set to be smaller than a width size and a length size of the heat transfer plate 91, respectively.

The heating element 922 is formed on one plate surface of the ceramic substrate 921 by vapor deposition or the like, and made of a conductive material, such as platinum. As illustrated in FIG. 4, the heating element 922 includes a pair of electrodes 9221 and a resistance pattern 9222.

The material of the heating element 922 is not limited to platinum, and a conductive material, such as stainless steel or tungsten, may be adopted. Further, the heating element 922 is not limited to a configuration that is formed on one plate surface of the ceramic substrate 921 by vapor deposition or the like, but it may be possible to adopt a configuration that is obtained by processing stainless steel or the like and bonding the processed one by thermal compression bonding.

The electrodes 9221 as a pair extend from one end side (a right end side in FIG. 4) to the other end side (a left end side in FIG. 4) of the ceramic substrate 921, and face each other along the width direction of the ceramic substrate 921.

The resistance pattern 9222 is connected (electrically connected) to one of the electrodes 9221 at one end thereof, extends from the one end along a U-shape so as to conform to an outer edge shape of the ceramic substrate 921 while being curved in a wavelike form with a predetermined line width, and is connected (electrically connected) to the other one of the electrodes 9221 at the other end thereof.

The resistance pattern 9222 generates heat by applying a voltage (supplying electricity) to the pair of electrodes 9221 (generates thermal energy).

A bonding metal layer (not illustrated) constituted of a multi-layer membrane made of titanium, platinum, and gold for example is provided on the entire surface of the other plate surface of the ceramic substrate 921 described above (a plate surface on which the heating element 922 is not provided). The ceramic heater 92 is fixed to a back surface of the treatment surface 911 of the heat transfer plate 91 so as to be located in an approximately central portion in the width direction, by soldering the back surface and the bonding metal layer through AuSn solder.

The wire relay unit 93 is configured with a flexible board and electrically connects the pair of lead wires 94 (conductors 941) included in the electrical cable C and the pair of electrodes 9221. As illustrated in FIG. 5 or FIG. 6, the wire relay unit 93 includes a substrate 931 and a pair of wiring patterns 932. In FIG. 5, for simplicity of explanation, the substrate 931 is indicated by a chain line.

The substrate 931 is an elongated sheet made of an insulating material, such as polyimide.

Here, a width size of the substrate 931 is set to be approximately equal to a width size of the ceramic substrate 921.

Each of the wiring patterns 932, as a pair, is formed on one surface of the substrate 931 by vapor deposition or the like. In the first embodiment, the same material (copper) as the conductors 941 of the lead wires 94 is adopted as a material of the wiring patterns 932. The wiring patterns 932 as a pair extend from one end side (a left end side in FIG. 5 and FIG. 6) to the other end side (a right end side in FIG. 5 and FIG. 6) of the substrate 931, and face each other along the width direction of the substrate 931.

One ends (left end sides in FIG. 6) of the wiring patterns 932 as a pair are connected (bonded) to the electrodes 9221 as a pair via a bonding layer BL1 (FIG. 6) made of a conductive material, such as gold, AuSn, or silver. Further, the conductors 941 of the lead wires 94 as a pair are connected (bonded) to the other ends (right end sides in FIG. 6) of the wiring patterns 932 as a pair via a bonding layer BL2 (FIG. 6) made of a conductive material, such as gold, AuSn, or silver.

An insulating sheet 933 made of polyimide or the like is attached to the surface of the substrate 931, on which the wiring patterns 932 as a pair are provided, so as to cover exposed portions of the wiring patterns 932 as a pair as illustrated in FIG. 6.

The conductors 941 and the wiring patterns 932 as described above serve as an electrical conduction path from the control device 3 to the ceramic heater 92, and have functions as a wiring unit 10 (FIG. 5 and FIG. 6) according to the present disclosure.

Here, while the conductors 941 and the wiring patterns 932 are made of the same material (copper) as described above, areas of cross sections of the wiring patterns 932 cut along a cutting plane along the width direction are set to be smaller than areas of cross sections of the conductors 941 (areas of cross sections cut along a cutting plane perpendicular to a longitudinal direction). Therefore, thermal resistance of the wiring patterns 932 per unit length in the longitudinal direction is higher than thermal resistance of the conductors 941 per unit length in the longitudinal direction. That is, the wiring patterns 932 have functions as a high thermal resistance unit according to the present disclosure. Further, the conductors 941 have functions as a low thermal resistance unit according to the present disclosure.

It is preferable to set length sizes of the wiring patterns 932 in the longitudinal direction to a requisite minimum length (for example, about 10 millimeters (mm)) in consideration of thermal resistance and electrical resistance of the wiring patterns 932.

Configuration of Control Device and Foot Switch

The foot switch 4 is a part to be operated with an operator's foot. Electrical conduction to the ceramic heater 92 (the resistance pattern 9222) is turned on and off in accordance with operation on the foot switch 4.

A means for turning on and off the electrical conduction is not limited to the foot switch 4, but a different switch, such as a manual switch, may be adopted.

The control device 3 is configured to include a central processing unit (CPU) or the like, and comprehensively controls operation of the energy treatment tool 2. More specifically, the control device 3 applies a voltage to the ceramic heater 92 (the pair of electrodes 9221) (conducts electricity to the resistance pattern 9222) via the pair of lead wires 94 and the wire relay unit 93 (the pair of wiring patterns 932) in accordance with operation on the foot switch 4 performed by the operator (operation of turning on the electrical conduction), to thereby heat the heat transfer plate 91.

Operation of Treatment System

Next, operation of the treatment system 1 described above will be explained.

The operator grips the energy treatment tool 2 and inserts the tip portion of the energy treatment tool 2 (the grasping unit 7 and a part of the shaft 6) into an abdominal cavity through an abdominal wall using a trocar or the like, for example. Then, the operator operates the operation knob 51 and grasps treatment target body tissue by the grasping unit 7.

Subsequently, the operator operates the foot switch 4 and turns on the electrical conduction from the control device 3 to the energy treatment tool 2 (the ceramic heater 92). When the electrical conduction is turned on, the control device 3 applies a voltage to the pair of electrodes 9221 via the pair of lead wires 94 and the wire relay unit 93, to thereby heat the heat transfer plate 91. Then, the body tissue in contact with the heat transfer plate 91 is treated with the heat of the heat transfer plate 91.

In the energy treatment tool 2 according to the first embodiment described above, the ceramic heater 92 (the pair of electrodes 9221) and the pair of lead wires 94 are connected by the pair of wiring patterns 932 that have higher thermal resistance than the lead wires 94 (the conductors 941). That is, the energy treatment tool 2 is configured such that heat generated by the ceramic heater 92 is less likely to flow to the lead wires 94. Therefore, the heat transfer plate 91 does not include a portion where temperature is locally reduced, so that temperature of the treatment surface 911 can be equalized.

Thus, according to the energy treatment tool 2 of the first embodiment, it is possible to achieve an effect that thermal energy is uniformly applied to body tissue. Further, it is configured that heat generated by the ceramic heater 92 is less likely to flow to the lead wires 94, so that it is not necessary to improve heat resistance of the lead wires 94 and it becomes possible to use the lead wires 94 of less expensive models. Furthermore, by adopting the energy treatment tool 2 as described above, it becomes possible to heat the heat transfer plate 91 to target temperature in a shorter time and treat body tissue in a shorter time as compared to a conventional configuration.

Moreover, in the energy treatment tool 2 according to the first embodiment, the wiring patterns 932 are configured with thin membranes formed on the substrate 931 by vapor deposition, and have smaller cross-sectional areas than those of the conductors 941. Therefore, it is possible to easily manufacture the high thermal resistance unit according to the present disclosure with higher thermal resistance than the conductors 941, with a simple structure. Furthermore, by configuring the high thermal resistance unit according to the present disclosure using the wiring patterns 932 formed on the flexible board (the wire relay unit 93), it is possible to reduce the size of the energy treatment tool 2 without any disturbance even when the high thermal resistance unit is provided on the energy treatment tool 2.

Incidentally, as the energy generating unit according to the present disclosure, it may be possible to adopt a sheet heater, in which a resistance pattern is provided on a substrate made of an insulating material, such as polyimide (for example, see JP 2015-208415 A), instead of the ceramic heater 92. However, because the insulating material, such as polyimide, is used in the substrate of the sheet heater as described above, it is difficult to heat the heat transfer plate 91 to a high temperature range that is realized by the ceramic heater 92.

In contrast, in the energy treatment tool 2 according to the first embodiment, the ceramic heater 92 is adopted as the energy generating unit according to the present disclosure. Therefore, it is possible to heat the heat transfer plate 91 to a higher temperature range and treat body tissue in a shorter time as compared to the sheet heater as described above. In particular, while it is likely that the temperature of the treatment surface 911 extremely fluctuates due to flow of heat to the pair of lead wires 94 when the heat transfer plate 91 is heated to the high temperature range as described above, the temperature of the treatment surface 911 can be equalized by placing the wire relay unit 93 between the ceramic heater 92 and the pair of lead wires 94 as in the first embodiment.

### Second Embodiment

Next, a second embodiment will be described.

In the description of the second embodiment, the same components as those of the first embodiment described above are denoted by the same reference signs, and detailed explanation thereof will be omitted or simplified.

FIG. 7 is a diagram illustrating a configuration of an energy applying structure 9A according to the second embodiment. Specifically, FIG. 7 is a side view corresponding to FIG. 6.

In the energy applying structure 9A according to the second embodiment, as illustrated in FIG. 7, a wire relay unit 93A is adopted instead of the wire relay unit 93 of the energy applying structure 9 explained in the first embodiment described above (FIG. 3 to FIG. 6). An energy applying structure (not illustrated) supported by the holding member 8′ has the same configuration as the energy applying structure 9A that is supported by the holding member 8.

Specifically, as illustrated in FIG. 7, a pair of wiring patterns 932A, which are made of a material different from that of the wiring patterns 932 (a material different from that of the conductors 941), is adopted in the wire relay unit 93A, instead of the pair of wiring patterns 932 of the first embodiment.

In the second embodiment, nickel is adopted as the material of the wiring patterns 932A. Thermal conductivity of nickel is about one-fifth of thermal conductivity of copper (the conductors 941). Therefore, thermal resistance of the wiring patterns 932A per unit length in the longitudinal direction is higher than thermal resistance of the conductors 941 per unit length in the longitudinal direction, similarly to the first embodiment described above. That is, the wiring patterns 932A have functions as the high thermal resistance unit according to the present disclosure. Further, the conductors 941 and the wiring patterns 932A have functions as a wiring unit 10A (FIG. 7 according to the present disclosure.

Here, electrical resistivity of nickel is about four times of electrical resistivity of copper. Therefore, in the second embodiment, thicknesses of the wiring patterns 932A are increased as compared to the wiring patterns 932 explained in the first embodiment described above, in order to reduce electrical resistance of the wiring patterns 932A (FIG. 7).

The material of the wiring patterns 932A is not limited to nickel, but a different material may be used as long as the material has lower thermal conductivity than the conductors 941.

As in the energy applying structure 9A according to the second embodiment as described above, even when the wiring patterns 932A are made of a material that is different from the material of the conductors 941, it is possible to achieve the same effect as that of the first embodiment described above.

### Third Embodiment

Next, a third embodiment will be described.

In the description of the third embodiment, the same components as those of the first embodiment described above are denoted by the same reference signs, and detailed explanation thereof will be omitted or simplified.

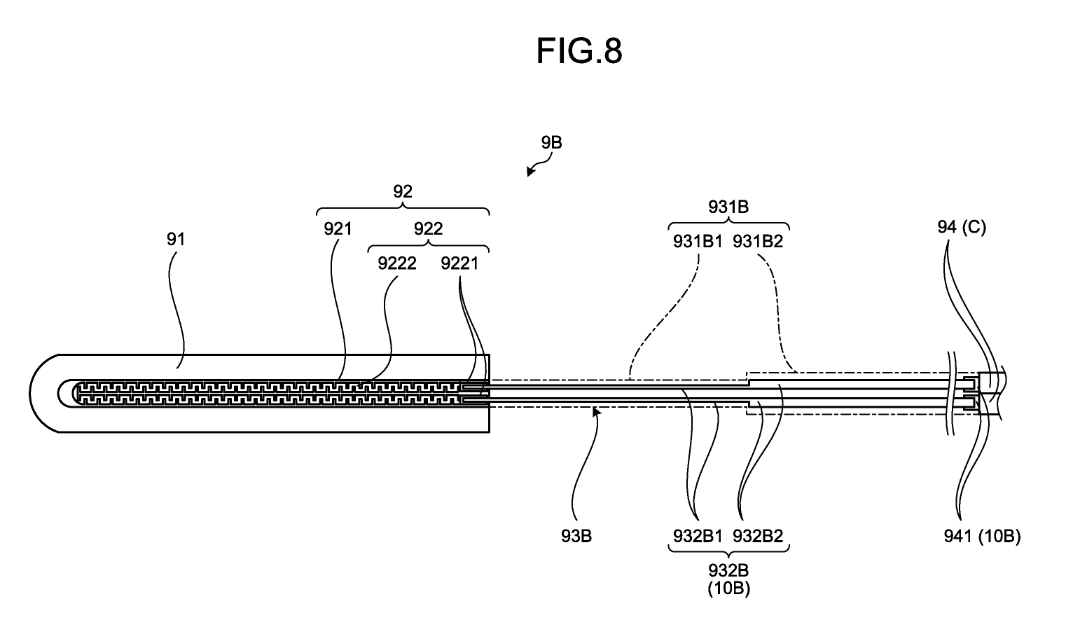

FIG. 8 is a diagram illustrating a configuration of an energy applying structure 9B according to the third embodiment. Specifically, FIG. 8 is a diagram corresponding to FIG. 5.

In the energy applying structure 9B according to the third embodiment, as illustrated in FIG. 8, a wire relay unit 93B is adopted instead of the wire relay unit 93 of the energy applying structure 9 (FIG. 3 to FIG. 6) explained in the first embodiment described above. An energy applying structure (not illustrated) supported by the holding member 8′ has the same configuration as the energy applying structure 9B that is supported by the holding member 8.

Specifically, the wire relay unit 93B is configured with a flexible board similarly to the wire relay unit 93 explained in the first embodiment described above, but includes a substrate 931B and a pair of wiring patterns 932B that have different shapes from those of the substrate 931 and the pair of wiring patterns 932. In FIG. 8, for simplicity of explanation, the substrate 931B is indicated by a chain line.

As illustrated in FIG. 8, the substrate 931B has a shape in which a width size of one end side (a left end side in FIG. 8) is smaller than a width size of the other end side (a right end side in FIG. 8). Hereinafter, a region on the one end side with a smaller width size will be referred to as a substrate narrow-width region 931B1 and a region on the other end side with a large width size will be referred to as a substrate wide-width region 931B2.

The substrate narrow-width region 931B1 has approximately the same width size as the width size of the ceramic substrate 921, and extends along a longitudinal direction (in a horizontal direction in FIG. 8).

The substrate wide-width region 931B2 is extended outward in a width direction from a position of a boundary with the substrate narrow-width region 931B1, has a wider width size than the substrate narrow-width region 931B1, and extends along the longitudinal direction.

As illustrated in FIG. 8, the wiring patterns 932B as a pair have shapes in which width sizes on one end side (a left end side in FIG. 8) are smaller than width sizes on the other end side (a right end side in FIG. 8) in conformity with the shape of the substrate 931B. Hereinafter, regions on the one end side with smaller width sizes will be referred to as pattern narrow-width regions 932B1 and regions on the other end side with wider width sizes will be referred to as pattern wide-width regions 932B2.

The pattern narrow-width regions 932B1 as a pair have constant width sizes and extend from one end side (a left end side in FIG. 8) of the substrate narrow-width region 931B1 to the vicinity of the position of the boundary between the substrate narrow-width region 931B1 and the substrate wide-width region 931B2 along the longitudinal direction. The pattern narrow-width regions 932B1 as a pair face each other along the width direction of the substrate 931B.

The pattern wide-width regions 932B2 as a pair extend outward in the width direction from positions of boundaries with the pattern narrow-width regions 932B1 as a pair in conformity with an outer shape of the substrate wide-width region 931B2, have wider width sizes than the width sizes of the pattern narrow-width regions 932B1, and extend along the longitudinal direction. The pattern wide-width regions 932B2 as a pair face each other along the width direction of the substrate 931B.

The pattern wide-width regions 932B2 have approximately the same thickness sizes as thickness sizes of the pattern narrow-width regions 932B1.

As illustrated in FIG. 8, one end sides (left end sides in FIG. 8) of the pattern narrow-width regions 932B1 as a pair are connected (bonded) to the electrodes 9221 as a pair via a bonding layer (not illustrated) made of a conductive material, such as gold, AuSn, or silver. Further, the conductors 941 of the lead wires 94 as a pair are connected (bonded) to the other end sides (right end sides in FIG. 8) of the pattern wide-width regions 932B2 as a pair via a bonding layer (not illustrated) made of a conductive material, such as gold, AuSn, or silver.

While detailed illustration is omitted, an insulating sheet made of polyimide or the like is attached to the surface of the substrate 931, on which the wiring patterns 932B as a pair are provided, so as to cover exposed portions of the wiring patterns 932B as a pair, similarly to the first embodiment described above.

Here, areas of cross sections of the pattern wide-width regions 932B2 cut along a cutting plane along the width direction are set to be smaller than the areas of the cross sections of the conductors 941(the areas of the cross sections cut along a cutting plane perpendicular to the longitudinal direction). Further, areas of cross sections of the pattern narrow-width regions 932B1 cut along a cutting plane along the width direction are set to be smaller than the areas of the cross sections of the pattern wide-width regions 932B2 cut along a cutting plane along the width direction. Therefore, the thermal resistance per unit length in the longitudinal direction is reduced in the following order: the lowest the pattern narrow-width regions 932B1, the pattern wide-width regions 932B2, and the conductors 941. That is, the wiring patterns 932B have functions as the high thermal resistance unit according to the present disclosure. Further, the conductors 941 and the wiring patterns 932B have functions as a wiring unit 10B (FIG. 8) according to the present disclosure.

As in the energy applying structure 9B according to the third embodiment as described above, even when the wiring patterns 932B are constituted of the pattern narrow-width regions 932B1 and the pattern wide-width regions 932B2, it is possible to achieve the same effect as that of the first embodiment described above.

### Fourth Embodiment

Next, a fourth embodiment will be described.

In the description of the fourth embodiment, the same components as those of the first embodiment described above are denoted by the same reference signs, and detailed explanation thereof will be omitted or simplified.

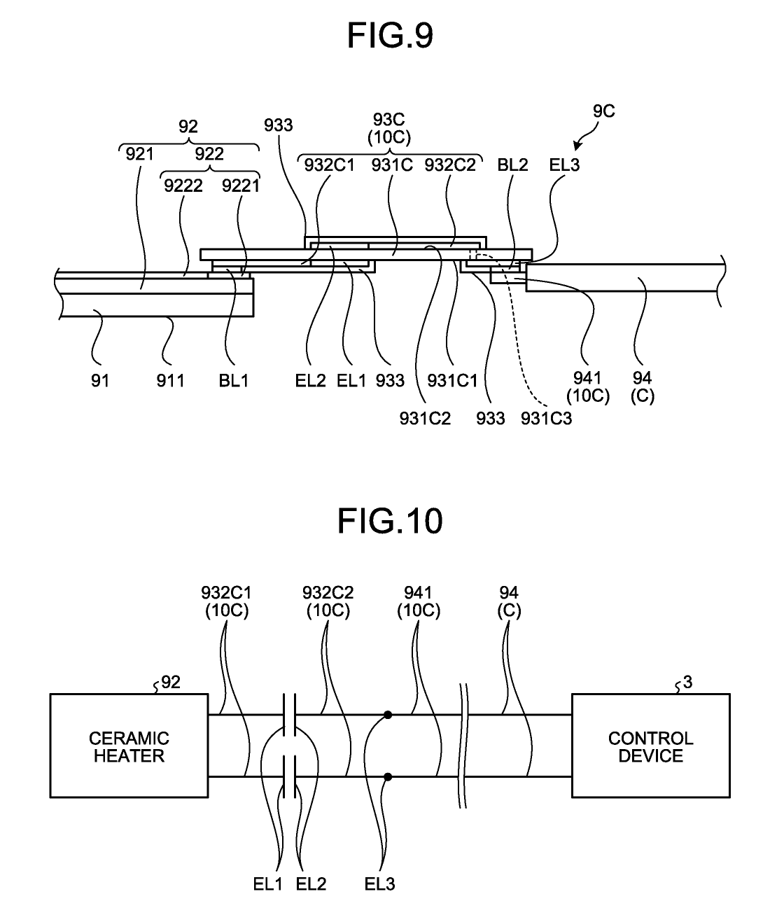

FIG. 9 is a diagram illustrating a configuration of an energy applying structure 9C according to the fourth embodiment. Specifically, FIG. 9 is a side view corresponding to FIG. 6. FIG. 10 is a diagram illustrating a circuit model of a wire relay unit 93C.

In the energy applying structure 9C according to the fourth embodiment, as illustrated in FIG. 9, the wire relay unit 93C is adopted instead of the wire relay unit 93 of the energy applying structure 9 explained in the first embodiment described above (FIG. 3 to FIG. 6). An energy applying structure (not illustrated) supported by the holding member 8′ has the same configuration as the energy applying structure 9C that is supported by the holding member 8.

Specifically, the wire relay unit 93C is configured with a flexible board similarly to the wire relay unit 93 explained in the first embodiment described above. As illustrated in FIG. 9, the wire relay unit 93C includes a substrate 931C, a pair of first wiring patterns 932C1, and a pair of second wiring patterns 932C2.

The substrate 931C is an elongated sheet made of an insulating material, such as polyimide (a dielectric material), similarly to the substrate 931 explained in the first embodiment described above.

The first wiring patterns 932C1 as a pair are formed on one surface 931C1 of the substrate 931C by vapor deposition or the like, and have functions as a wire according to the present disclosure. The first wiring patterns 932C1 as a pair extend from one end side (a left end side in FIG. 9) of the substrate 931C to an approximately central portion of the substrate 931C in a longitudinal direction, and face each other along a width direction of the substrate 931C.

First electrodes EL1 as a pair (FIG. 9) are provided on one ends (right ends in FIG. 9) of the first wiring patterns 932C1 as a pair.

The second wiring patterns 932C2 as a pair are formed on another surface 931C2 of the substrate 931C by vapor deposition or the like, and have functions as a wire according to the present disclosure. The second wiring patterns 932C2 as a pair extend from the other end side (a right end side in FIG. 9) of the substrate 931C to an approximately central portion of the substrate 931C in the longitudinal direction, and face each other along the width direction of the substrate 931C.

Second electrodes EL2 as a pair (FIG. 9) are provided on one ends (left ends in FIG. 9) of the second wiring patterns 932C2 as a pair, so as to face the first electrodes EL 1 as a pair.

Further, through holes 931C3 (FIG. 9) are provided at positions on the other end sides (right end sides in FIG. 9) of the second wiring patterns 932C2 as a pair in the substrate 931C. Furthermore, third electrodes EL3 as a pair (FIG. 9), which are electrically connected to the second wiring patterns 932C2 as a pair via the through holes 931C3, are provided on the one surface 931C1 of the substrate 931C.

The other end sides (left end sides in FIG. 9) of the first wiring patterns 932C1 as a pair are connected (bonded) to the electrodes 9221 as a pair via the bonding layer BL1 (FIG. 9). Further, the conductors 941 of the lead wires 94 as a pair are connected (bonded) to the third electrodes EL3 as a pair via the bonding layer BL2 (FIG. 9).

An insulating sheet 933 made of polyimide or the like is attached to each of the surfaces 931C1 and 931C2 of the substrate 931C so as to cover exposed portions of the pair of first wiring patterns 932C1, the pair of electrodes EL1, the pair of second wiring patterns 932C2, the pair of second electrodes EL2, and the pair of third electrodes EL3, similarly to the first embodiment described above.

With the configuration as described above, as illustrated in FIG. 10, when an alternating current (with a frequency of a few MHz to a few dozen MHz, for example) is supplied from the control device 3 to the pair of third electrodes EL2 via the pair of lead wires 94, the first electrodes EL1 facing each other and the second electrodes EL2 facing each other are connected in a non-contact manner by capacitive coupling, so that electricity is supplied to the ceramic heater 92 (the resistance pattern 9222).

That is, the conductors 941, the pair of first wiring patterns 932C1, and the pair of second wiring patterns 932C2 serve as an electrical conduction path from the control device 3 to the ceramic heater 92, and have functions as a wiring unit 10C (FIG. 9 and FIG. 10) according to the present disclosure.

Here, the first wiring patterns 932C1 and the second wiring patterns 932C2 functioning as the electrical conduction path are not connected (electrically connected) to each other because the substrate 931C that is made of an insulating material with low thermal conductivity is arranged between them. Therefore, thermal resistance of the electrical conduction path (the first wiring patterns 932C1, the substrate 931C, and the second wiring patterns 932C2) is higher than the thermal resistance of the conductors 941. That is, the electrical conduction path has a function as the high thermal resistance unit according to the present disclosure.

As in the energy applying structure 9C according to the fourth embodiment as described above, even when the first wiring patterns 932C1 and the second wiring patterns 932C2 are connected in a non-contact manner by capacitive coupling, it is possible to achieve the same effect as that of the first embodiment described above.

### Other Embodiments

While the embodiments for carrying out the present disclosure have been described above, the present disclosure is not limited only by the first to fourth embodiments.

In the first to fourth embodiments described above, the shape of the energy treatment tool 2 is not limited to the shape explained in the first to fourth embodiments described above, but a different shape, such as a shape like a forceps, may be adopted as long as the same functions are implemented. Further, the shaft 6 may be formed in a curved shape.

In the first to fourth embodiments described above, the energy applying structures 9 and 9′ (9A to 9C) are provided on both of the holding members 8 and 8′, but the configuration is not limited thereto. It may be possible to adopt a configuration in which the energy applying structure is provided on only one of the holding members 8 and 8′.

In the first to fourth embodiments described above, the energy applying structures 9 and 9′(9A to 9C) are configured to apply thermal energy to body tissue, but the configuration is not limited thereto. It may be possible to apply high-frequency energy or ultrasonic energy other than the thermal energy.

In the third embodiment described above, it may be possible to remove the pair of lead wires 94, and connect the ceramic heater 92 and the control device 3 by only the wire relay unit 93B. In this configuration, the pattern narrow-width regions 932B1 have functions as the high thermal resistance unit according to the present disclosure and the pattern wide-width regions 932B2 have functions as the low thermal resistance unit according to the present disclosure in the wire relay unit 93B.

According to the energy treatment tool, it is possible to uniformly apply thermal energy to body tissue to be treated.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment tool comprising:
a heat transfer plate including a treatment surface for treating body tissue;
an energy generating unit configured to generate thermal energy and transmit the thermal energy to the heat transfer plate; and
a wiring unit connected to the energy generating unit and serving as an electrical conduction path to the energy generating unit, the wiring unit including:
a low thermal resistance unit; and
a high thermal resistance unit that has higher thermal resistance than the low thermal resistance unit and connects the low thermal resistance unit and the energy generating unit.

2. The energy treatment tool according to claim 1, wherein the high thermal resistance unit includes a region with a smaller cross-sectional area than the low thermal resistance unit.

3. The energy treatment tool according to claim 1, wherein the high thermal resistance unit is made of a material with lower thermal conductivity than the low thermal resistance unit.

4. The energy treatment tool according to claim 1, wherein the high thermal resistance unit includes a plurality of wires that are connected to each other in a non-contact manner by capacitive coupling.

5. The energy treatment tool according to claim 1, wherein
the energy generating unit is configured with a ceramic heater, the ceramic heater including:
a ceramic substrate, one plate surface of which is bonded to the heat transfer plate; and
a heating element provided on another plate surface of the ceramic substrate and configured to generate thermal energy by electrical conduction.

6. The energy treatment tool according to claim 1, wherein
the low thermal resistance unit is configured with a lead wire, and
the high thermal resistance unit is configured with a wiring pattern provided on a flexible board.
